(19) United States
(12) Patent Application Publication
(10) Pub. No.: US 2007/0088022 A1
(43) Pub. Date: Apr. 19, 2007

(54) Tetrahydroquinolines, synthesis thereof, and intermediates thereto

(75) Inventor: Gregg B. Feigelson, Chester, NY (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/581,638

(22) Filed: Oct. 16, 2006

Related U.S. Application Data
(60) Provisional application No. 60/727,606, filed on Oct. 17, 2005.

(51) Int. Cl. A61K 31/551; A61K 31/498; C07D 487/04

(52) U.S. Cl. 514/219; 540/472; 540/494; 514/249; 544/343

(57) ABSTRACT

The present invention relates to methods for synthesizing compounds useful as 5HT_{2C} agonists or partial agonists, derivatives thereof, and to intermediates thereto.

### CROSS-REFERENCE TO RELATED APPLICATIONS

This application claims priority to U.S. provisional patent application Ser. No. 60/727,606, filed Oct. 17, 2005, the entirety of which is hereby incorporated herein by reference.

### FIELD OF THE INVENTION

The present invention relates to methods for synthesizing compounds useful as 5HT_{2C} agonists or partial agonists, derivatives thereof, and to intermediates thereto.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et al., Psychopharmacology 124: 57-73, 1996; Cowen, P. J., et al., Human Psychopharmacology 10: 385-391, 1995; Rosenzweig-Lipson, S., et al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et al., Neuropharmacology 37: 265-272, 1998; Fox, S. H., et al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et al., Neuropharmacology 37: 953-955, 1998; Di Matteo, V., et al., Neuropharmacology 38: 1195-1205, 1999; Di Giovanni, G., et al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

As described herein, the present invention provides methods for preparing compounds having activity as 5HT_{2C} agonists or partial agonists. These compounds are useful for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, gastrointestinal disorders, obesity, or a central nervous system deficiency associated with trauma, stroke, or spinal cord injury. Such compounds include those of formula I: 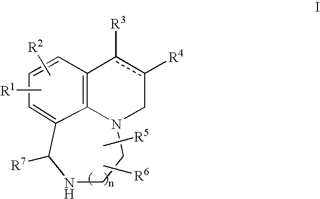
or a pharmaceutically acceptable salt thereof, wherein:

[symbol] designates a single or double bond;

n is 0, 1, or 2;

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

R³ and R⁴ are taken together to form a saturated or partially unsaturated 4-8-membered ring, wherein said ring is optionally substituted with 1-3 groups independently selected from halogen, —R, or —OR; and

R⁵, R⁶, and R⁷ are each independently —R.

The present invention also provides synthetic intermediates useful for preparing such compounds.

### DETAILED DESCRIPTION OF CERTAIN EMBODIMENTS

The methods and intermediates of the present invention are useful for preparing compounds as described in, e.g. International Patent Application No. PCT/US03/12747 (International Publication No. WO 03/091250), in the name of Ramamoorthy, the entirety of which is incorporated herein by reference. In certain embodiments, the present compounds are generally prepared according to Scheme I set forth below:

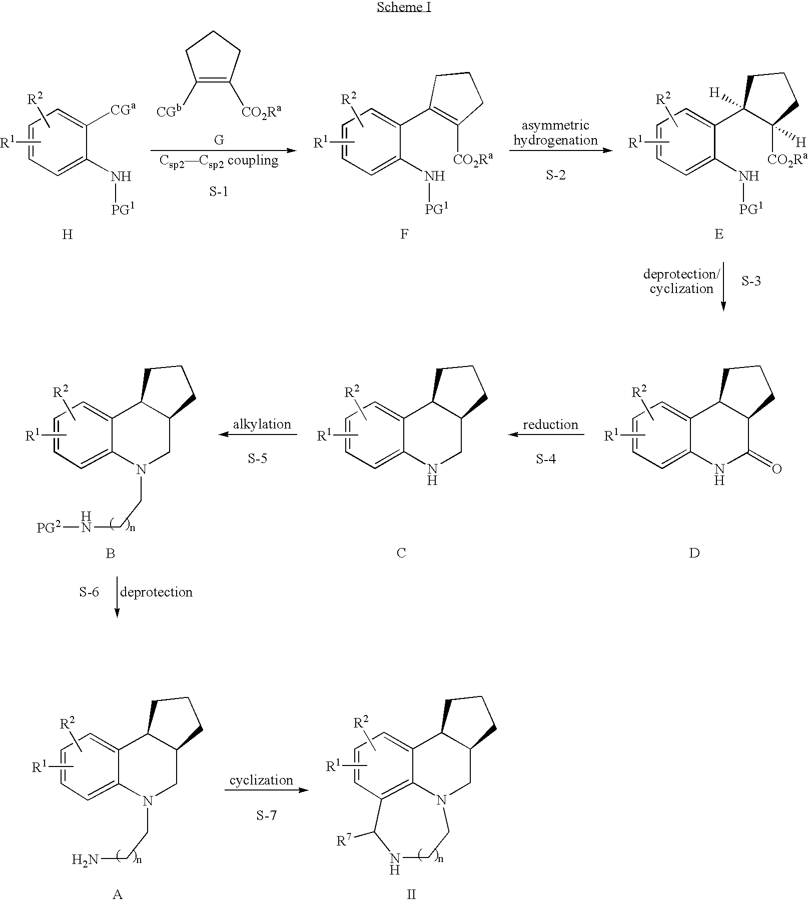

In Scheme I above, each R^{a} is independently hydrogen or a suitable carboxyl protecting group and each of n, PG¹, PG² R¹, R², R⁷, CG^{a}, and CG^{b} is as defined below and in classes and subclasses as described herein.

In one aspect, the present invention provides methods for preparing chiral cis-1,2-disubstituted cyclopentane compounds of formulae E, D, C, B, A, and II in enantiomerically enriched form according to the steps depicted in Scheme I, above. In compounds of the present formulae, R¹ and R² are as defined above for compounds of formula I and are each independently halogen, —CN, phenyl, —R, —OR, —C₁₋₆ perfluoroalkyl, or —OC₁₋₆ perfluoroalkyl, wherein each R is independently hydrogen or a C₁₋₆ alkyl group.

At step S-1, a compound of formula H is coupled to a compound of formula G, via a Cₛₚ₂-Cₛₚ₂ coupling reaction between the carbon centers bearing complementary coupling groups CG^{a} and CG^{b}. Suitable coupling reactions are well known to one of ordinary skill in the art and typically involve one of the coupling groups being an electron-withdrawing group (e.g., Cl, Br, I, OTf, etc.), such that the resulting polar carbon-CG bond is susceptible to oxidative addition by an electron-rich metal (e.g., a low-valent palladium species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-IV} species or a Ni^{II-IV} species). Exemplary reactions include those described in Metal-Catalyzed Cross-Coupling Reactions, A. de Meijere and F. Diederich, Eds., 2^{nd} Edition, John Wiley & Sons, 2004. In certain embodiments, CG^{a} in compounds of formula H is a boronic acid, a boronic ester, or a borane. In other embodiments, CG^{a} in compounds of formula H is a boronic ester. In still other embodiments, CG^{a} in compounds of formula H is a boronic acid. In certain embodiments, CG^{b} in compounds of formula G is Br, I, or OTf. In other embodiments, CG^{b} in compounds of formula G is OTf. In certain embodiments, the transformation is catalyzed by a palladium species. In other embodiments, the reaction is performed as described in Jaroch et al., U.S. Pat. No. 6,391,887, Example 15.

The PG¹ group of formulae H, F, and E is a suitable amino protecting group. Suitable amino protecting groups are well known in the art and include those described in detail in Protecting Groups in Organic Synthesis, T. W. Greene and P. G. M. Wuts, 3^{rd} Edition, John Wiley & Sons, 1999, the entirety of which is incorporated herein by reference. Suitable amino protecting groups, taken with the —NH— moiety to which they are attached, include, but are not limited to, aralkylamines, carbamates, allyl amines, amides, and the like. Examples of PG¹ groups of formulae H, F, and E include t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, pivaloyl and the like. In certain embodiments, the PG¹ group in compounds of formulae H, F, and E is t-butyloxycarbonyl, ethyloxycarbonyl, pivaloyl, or acetyl. In other embodiments, the PG¹ group in compounds of formulae H, F, and E is pivaloyl.

As defined generally above, the R^{a} group of formulae G, F, and E is, independently for each formula, either hydrogen or a suitable carboxyl protecting group. Suitable carboxyl protecting groups are well known in the art and include those described in detail in Greene (1999). Suitable carboxyl protecting groups, taken with the carboxylate moiety to which they are attached, include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters. In certain embodiments, the R^{a} group of any of formulae G, F, or E is methyl, ethyl, or benzyl. In other embodiments, the R^{a} group of any of formulae G, F, or E is ethyl.

At step S-2, the cyclopentenyl olefin of formula F is hydrogenated in an asymmetric fashion to provide chiral cyclopentenes of formula E in enantiomerically enriched form. As used herein, the term “enantiomerically enriched,” signifies that the ratio of enantiomers present in a mixture thereof is other than 1:1. In certain embodiments, the asymmetric hydrogenation is catalyzed by a suitable chiral catalyst. In certain embodiments, the chiral catalyst is a complex comprising a transition metal species and a suitable chiral ligand. In certain embodiments, the transition metal species is a late transition metal species (e.g., a Ru, Rh, Pd, Ir, or Pt species). In other embodiments the transition metal species is a rhodium or ruthenium species. In certain embodiments, the chiral ligand contains a phosphorus moiety that is capable of binding a transition metal species (e.g., a phosphine or phosphite moiety). In other embodiments the chiral ligand contains an olefinic moiety that is capable of binding a transition metal species. In yet other embodiments, the chiral ligand contains a carbene moiety that is capable of binding to a transition metal species. Suitable chiral ligands for asymmetric hydrogenation are well known in the art; see, e.g., Stereochemistry of Organic Compounds, E. L. Eliel and S. H. Silen, 1994, John Wiley and Sons; Asymmetric Catalysis in Organic Synthesis, R. Noyori, 1994, John Wiley and Sons; X. Cui and K. Burgess, Chem. Rev. 105:3272-3296 (2005); and W. Tang and X. Zhang, Chem. Rev. 103:3029-3069 (2003). Additional exemplary chiral ligands include, but are not limited to, JosiPhos-type, MandyPhos™-type, WalPhos-type, TaniaPhos™-type, RoPhos-type, DIPAMP-type, Butiphane-type, BPE-type, QUINAP-type, BINAP-type, NorPhos-type, MonoPhos™-type, TunePhos-type, MalPhos-type, DuPhos-type, PHOX-type, KetalPhos-type, f-KetalPhos-type, TangPhos-type, BIPHEP-type, ferrotane-type, Binaphane-type, f-Binaphane-type, Binapine-type, FAP-type, MOP-type, DIOP-type, ChiraPhos-type, BPPM-type, and BICP-type.

The term “asymmetric hydrogenation,” as used herein refers to the hydrogenation of an achiral or chiral substrate which results in an enantiomerically enriched chiral product. In certain embodiments the asymmetric hydrogenation is catalyzed by a chiral transition metal-containing species.

One of ordinary skill in the art would recognize that the CO₂R^{a} moiety of formula F may be in protected form (i.e., wherein R^{a} is a suitable carboxyl protecting group) during hydrogenation, or may be deprotected to yield the free carboxylic acid (i.e., wherein R^{a} is hydrogen) prior to hydrogenation. One of ordinary skill in the art would also recognize that wherein R^{a} is a protecting group that may be cleaved by hydrogenation, that cleavage of R^{a} and asymmetric hydrogenation of the cyclopentenyl olefin may occur concomitantly.

One of ordinary skill in the art will recognize that the asymmetric hydrogenation can be adapted to provide compounds of formulae E, D, C, B, A, and II in enantiomerically enriched form with stereochemistry opposite to that depicted. In certain embodiments, one enantiomer of a compound of formulae E, D, C, B, A, and II is formed substantially free from other stereoisomers. “Substantially free,” as used herein, means that the compound is made up of a significantly greater proportion of one enantiomer. In other embodiments, at least about 98% by weight of a desired enantiomer is present. In still other embodiments of the invention, at least about 99% by weight of a desired enantiomer is present. Such enantiomers may be isolated from racemic mixtures by any method known to those skilled in the art, including high performance liquid chromatography (HPLC) and chiral salt resolution, or prepared by methods described herein.

At step S-3, an enantiomerically enriched compound of formula E is cyclized to form an enantiomerically enriched compound of formula D. Where the R^{a} group of formula E is a suitable carboxyl protecting group, one of ordinary skill in the art would recognize that, depending on the choice of PG¹ and R^{a}, cyclization may be performed prior to cleavage of one or both of these protecting groups. Similarly, cleavage of one or both of these protecting groups may occur concomitant with cyclization. Alternatively, cleavage of PG¹ may be performed following cyclization. One of ordinary skill in the art would also recognize that, depending on the presence or absence of, and the choice of, PG¹ and R^{a}, lactam formation may occur spontaneously. One of ordinary skill in the art would also appreciate that lactam formation may be induced thermally, with base catalysis, or with acid (Lewis or Bronsted) catalysis.

At step S-4, the lactam carbonyl moiety of formula D is reduced with a suitable reducing agent to provide compounds of formula C. Suitable reducing agents are well known to one of ordinary skill in the art and include various metal hydrides (e.g., aluminum hydrides, borohydrides, etc.) and the like. Conditions for promoting reductions of this type are well known in the art, e.g., see Comprehensive Organic Transformations, R. C. Larock, 2^{nd} Edition, John Wiley & Sons, 1999.

At step S-5, N-alkylation of compounds of formula C affords compounds of formula B, wherein n is 0, 1, or 2 and PG2 is a suitable amino protecting group. Suitable amino groups are well known to one of ordinary skill in the art and are as defined above for the PG¹ group in compounds of formulae H, F, and E. In certain embodiments, this N-alkylation is performed with 2-methyl-2-oxazoline in the presence of catalytic amount of acid to afford compounds of formula B, wherein n is 1 and PG² is acetyl.

At step S-6, removal of the PG² protecting group in compounds of formula B affords the diamino compounds of formula A, wherein n is 0, 1, or 2. In certain embodiments, the PG² group in compounds of formula B is removed by acidolysis (including acid-promoted hydrolysis). It will be appreciated that upon removal of the PG² group in compounds of formula B by treatment with acid, a salt comprising compounds of formula A and the acid employed in the deprotection is formed. For example, where the PG² group of compounds formula B is removed by treatment with an acid such as trifluoroacetic acid, then the resulting diamino compound would be formed as its trifluoroacetic acid salt. One of ordinary skill in the art would recognize that a wide variety of acids are useful for removing amino protecting groups that are acid-labile (see, e.g., Greene, 1999) and therefore a wide variety of salt forms of compounds of formula A are contemplated.

In other embodiments, the PG² group in compounds of formula B is removed by base hydrolysis. One of ordinary skill in the art would recognize that a wide variety of bases are useful for removing base-labile amino protecting groups (see, e.g., Greene, 1999).

At step S-7, a compound of formula A is treated with an aldehyde of formula R⁷CHO wherein R⁷ is hydrogen or a C₁₋₆ alkyl group, or with a suitable formaldehyde equivalent, to provide a compound of formula II, wherein n is 0, 1, or 2 and R⁷ is hydrogen or a C₁₋₆ alkyl group. Cyclizations of this type are well known in the art, e.g., see E. D. Cox and J. M. Cook, Chem. Rev. 95:1797-1842, 1995; M. Chrzanowska and M. D. Rozwadowska Chem. Rev. 104:3341-3370, 2004; J. Royer et al. Chem. Rev. 104:2311-2352, 2004; and B. E. Maryanoff et al. Chem. Rev. 104:1431-1628, 2004. One of ordinary skill in the art would recognize that a wide variety of reaction conditions are useful for promoting reactions of this type, therefore a wide variety of reaction conditions are contemplated. For example, the reaction may be conducted with or without thermal excitation, with or without acid catalysis (including both Lewis and Bronsted acids), with or without a means for removing or sequestering the water of condensation (e.g., Dean-Stark trapping, molecular sieves), and in protic or aprotic media.

One of ordinary skill in the art will recognize that suitable equivalents of formaldehyde for use in the above transformation include, but are not limited to, paraformaldehyde, dimethoxymethane, and 1,3,5-trioxane.

One of ordinary skill in the art will appreciate that a compound of formula II, as prepared by the methods of the present invention, may be treated with a suitable acid to form a salt thereof. In certain embodiments, a compound of formula II is treated with HCl to form the hydrochloride salt thereof.

According to one embodiment, the R¹ and R² groups of formulae H, F, E, D, C, B, A, and II are each independently an R group. According to another embodiment, one of the R¹ and R² groups of formulae H, F, E, D, C, B, A, and II is hydrogen. According to yet another embodiment, both of the R¹ and R² groups of formulae H, F, E, D, C, B, A, and II are hydrogen.

As used herein, the term “partially unsaturated” refers to a ring moiety that includes at least one double or triple bond between ring atoms. The term “partially unsaturated” is intended to encompass rings having multiple sites of unsaturation, but is not intended to include aromatic moieties. As used herein, the term “aromatic” designates compounds or moieties that, in accordance with the theory of Huickel, have a cyclic, delocalized 4n+2π-electron system.

According to another aspect, the present invention provides a method for preparing a compound of formula II: 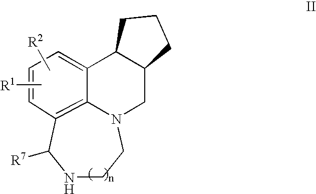
or a pharmaceutically acceptable salt thereof, wherein:

n is 0, 1, or 2;

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group; and

R⁷ is —R, comprising the steps of:
(a) Providing a Compound of Formula A: 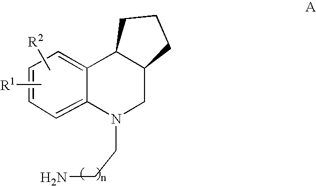
wherein:

n is 0, 1, or 2;

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, and

(b) Reacting Said Compound of Formula A with an Aldehyde of Formula R⁷CHO, or a Suitable Formaldehyde Equivalent, to Form a Compound of Formula II.

Cyclizations of this type are well known in the art, e.g., see E. D. Cox and J. M. Cook, Chem. Rev. 95:1797-1842, 1995; M. Chrzanowska and M. D. Rozwadowska Chem. Rev. 104:3341-3370, 2004; J. Royer et al. Chem. Rev. 104:2311-2352, 2004; and B. E. Maryanoff et al. Chem. Rev. 104:1431-1628, 2004. One of ordinary skill in the art would recognize that a wide variety of reaction conditions are useful for promoting reactions of this type, therefore a wide variety of reaction conditions are contemplated. In certain embodiments, the reaction is conducted with thermal excitation. In other embodiments, the reaction is conducted with acid catalysis. In still other embodiments, the reaction is conducted with a means for removing or sequestering the water of condensation (e.g., Dean-Stark trapping, molecular sieves).

According to one embodiment, step (b) above is performed using aqueous formaldehyde. In certain embodiments, aqueous formaldehyde is added in an amount sufficient to consume the compound of formula A. In certain embodiments, aqueous formaldehyde is added in amounts of at least about 0.90 mole equivalents, in amounts of about 0.90 mole equivalents to about 1.10 mole equivalents, or in amounts of from about 1.0 mole equivalents to about 1.05 mole equivalents relative to the compound of formula A.

According to another embodiment, step (b) is performed using a formaldehyde equivalent. In some embodiments, the formaldehyde equivalent is added in solid form to the reaction solvent to form a reaction suspension or the solid formaldehyde equivalent may be suspended in a reaction solvent and added to the reaction mixture. In other embodiments, paraformaldehyde is used as the formaldehyde equivalent, and is added in amounts sufficient to consume the compound of formula A. In some embodiments, paraformaldehyde is added in amounts of at least about 0.90 mole equivalents, in amounts of about 0.90 mole equivalents to about 1.10 mole equivalents, or in amounts of from about 1.0 mole equivalents to about 1.05 mole equivalents relative to the compound of formula A.

In certain embodiments, paraformaldehyde is in a solid form. Paraformaldehyde suitable for the reaction is commercially available in prills (or other granulated forms) and powders from a variety of suppliers such as Aldrich, Fluka, Celanese Chemicals, J. T. Baker, Mallinckrodt Laboratory Chemicals, Miljac Inc., Sego Int. Corp., Spectrum Chemicals Mfg., Total Specialty Chemicals Inc., US Chemicals Inc., Riedel-de Haen, Acros Organics, Pfaltz & Bauer Chemicals, Derivados, Lancaster Synthesis and EM Science. Certain suitable powder forms have at least about 10% particles retained on a 200 mesh screen.

As defined generally above for compounds of formula II, n is 0, 1, or 2. Accordingly, the present invention provides a method for preparing a compound of any of formulae IIa, IIb, or IIc: 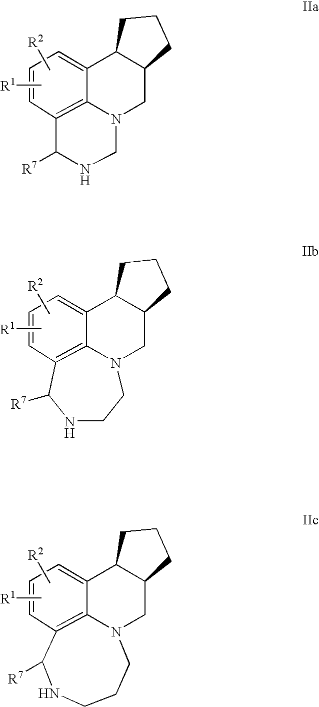
wherein each of R¹, R², R⁷, and n is as defined above and herein.

According to another embodiment, the present invention provides a method for preparing a compound of formula A: 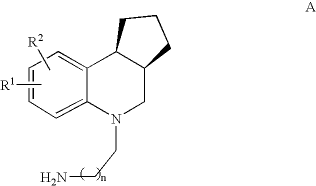
wherein:

n is 0, 1, or 2;

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, comprising the steps of:
(a) Providing a Compound of Formula B: 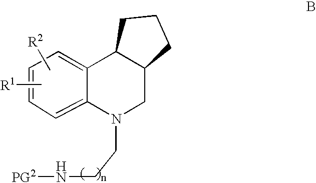
wherein:

n is 0, 1, or 2;

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group; and

PG² is a suitable amino protecting group, and

(c) Deprotecting said Compound of Formula B to Form Said Compound of Formula A.

As defined generally above, the PG² group in compounds of formula B is a suitable amino protecting group. Suitable amino protecting groups are well known in the art and include those described in detail in Greene (1999). Suitable amino protecting groups, taken with the —NH— moiety to which it is attached, include, but are not limited to, aralkylamines, carbamates, allyl amines, amides, and the like. In certain embodiments, the PG² group in compounds of formula B is selected from t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, or pivaloyl. In other embodiments, the PG² group in compounds of formula B is selected from acetyl, chloroacetyl, dichloroacetyl, trichloroacetyl, phenylacetyl, pivaloyl, or trifluoroacetyl. In still other embodiments, the amino protecting group in compounds of formula B is acetyl.

According to another embodiment, the present invention provides a method for preparing a compound of formula B: 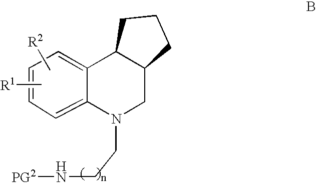
wherein:

n is 0, 1, or 2;

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group; and

PG² is a suitable amino protecting group, comprising the steps of:
(a) Providing a Compound of Formula C: 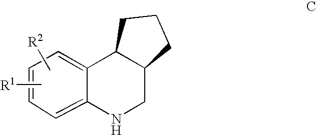
wherein:

R¹ and R² are each independently halogen, —CN, phenyl, —R, —OR, —C₁₋₆ perfluoroalkyl, or —O₁₋₆ perfluoroalkyl; and

each R is independently hydrogen or a C₁₋₆ alkyl group, and

(b) Alkylating Said Compound of Formula C to Form a Compound of Formula B.

In certain embodiments, the alkylation at step (b) above is achieved by reacting said compound of formula C with a compound of formula 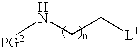
wherein said reaction is performed in a suitable medium and wherein:

n is 0, 1, or 2;

PG² is a suitable amino protecting group; and

L¹ is a suitable leaving group.

As defined above, L¹ is a suitable leaving group. Suitable leaving groups are well known in the art, e.g., see, Advanced Organic Chemistry, J. March, 5^{th} Edition, John Wiley and Sons, 2000. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties.

Examples of suitable leaving groups include chloro, iodo, bromo, fluoro, methanesulfonyloxy (mesyloxy), p-toluenesulfonyloxy (tosyloxy), trifluoromethanesulfonyloxy (triflyloxy), nitro-phenylsulfonyloxy (nosyloxy), and bromo-phenylsulfonyloxy (brosyloxy). In certain embodiments, L¹ is halogen. In other embodiments, L¹ is an optionally substituted alkylsulphonyloxy, optionally substituted alkenylsulfonyloxy, or optionally substituted arylsulfonyloxy.

According to an alternate embodiment, the suitable leaving group may be generated in situ within the reaction medium. That is, the L¹ moiety of compounds of formula 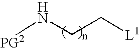
may be generated in situ from a suitable precursor. In situ generation of leaving groups from a variety of precursors is well known in the art, e.g., see, March (2000).

As defined generally above, the PG² group in compounds of formula 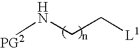
is a suitable amino protecting group. Suitable amino protecting groups are well known in the art and include those described in detail in Greene (1999). Suitable amino protecting groups, taken with the —NH— moiety to which it is attached, include, but are not limited to, aralkylamines, carbamates, allyl amines, amides, and the like. In certain embodiments, the PG² group in compounds of formula 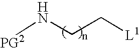
is selected from t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, or pivaloyl. In other embodiments, the PG² group in compounds of formula 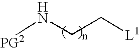
is selected from acetyl, chloroacetyl, dichloroacetyl, trichloroacetyl, phenylacetyl, pivaloyl, or trifluoroacetyl. In still other embodiments, the amino protecting group in compounds of formula 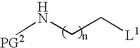
is acetyl.

In certain embodiments, the alkylation reaction at step (b) involves the displacement of a leaving group by the amino moiety in compounds of formula C. In certain embodiments, this alkylation reaction is conducted in the absence of any additional base. In certain embodiments, this alkylation reaction is conducted in the presence of a suitable base.

In certain embodiments, the alkylation reaction at step (b) is performed in a suitable medium. A suitable medium is a solvent or a solvent mixture that, in combination with the combined compounds, facilitates the progress of the reaction therebetween. The suitable solvent may solubilize one or more of the reaction components, or, alternatively, the suitable solvent may facilitate the suspension of one or more of the reaction components. Examples of suitable solvents useful in the present transformation are protic solvents (e.g., alcohols, water), halogenated hydrocarbons, ethers, esters, aromatic hydrocarbons, other polar or non-polar aprotic solvents, or mixtures of the aforementioned solvents. Such mixtures include, for example, mixtures of protic and aprotic solvents (e.g., benzene/methanol/water; benzene/water; dimethoxyethane/water, etc.). These and other such suitable solvents, and mixtures thereof, are well known in the art, e.g., see, March (2000).

According to yet another embodiment, one or more of the reagents may additionally serve as the suitable solvent. For example, an organic base such as triethylamine or diisopropylethylamine, if utilized in said reaction, may serve as the solvent in addition to its role as a basifying reagent.

In other embodiments, the alkylation at step (b) above is achieved by reacting said compound of formula C with 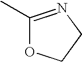
in the presence of a suitable acid to form a compound of formula B wherein n is 1 and PG² is acetyl. Such suitable acids are well known in the art and include inorganic acids, e.g. hydrochloric acid, hydrobromic acid, phosphoric acid, nitric acid, sulfuric acid or perchloric acid, or organic acids, e.g. acetic acid, oxalic acid, maleic acid, tartaric acid, citric acid, succinic acid, malonic acid, lower alkyl sulfonic acids, or aryl sulfonic acids. In certain embodiments, the alkylation at step (b) above is achieved by reacting said compound of formula C with 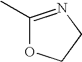
in the presence of para-toluenesulfonic acid.

In other embodiments, the alkylation of compounds of formula C to provide compounds of formula B is performed by means of a reductive alkylation procedure comprising imine formation and hydride reduction thereof by reacting the amino compounds of formula C with suitable carbonyl-containing compounds (typically aldehydes) and suitable reducing agents (e.g., NaBH₃CN, NaHB(OAc)₃). Reductive alkylation reactions of this type are well known in the art, e.g., see Larock (1999).

Yet another aspect of the present invention provides a method for preparing a compound of formula C: 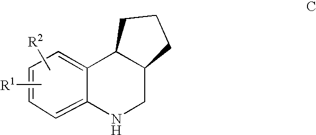
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, comprising the steps of:
(a) Providing a Compound of Formula D: 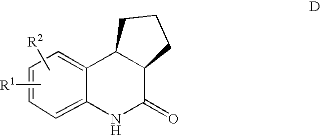
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, and

(b) Treating Said Compound of Formula D with a Suitable Reducing Agent to Form a Compound of Formula C.

Suitable reducing agents are well known to one of ordinary skill in the art and include various metal hydrides (e.g., aluminum hydrides, borohydrides, etc.) and the like. Conditions for promoting reductions of this type are well known in the are, e.g., see Larock, (1999). In certain embodiments, the reducing agent is selected from aluminum hydride species or borohydride species. In other embodiments, the reducing agent is selected from LiAlH₄, NaAlH₄, LiHAl(OMe)₃, BH₃, or NaBH₄. In still other embodiments the reducing agent is LiAlH₄.

According to another embodiment, the present invention provides a method of obtaining a compound of formula D: 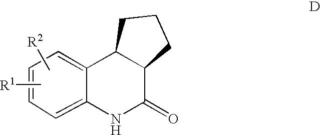
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, comprising the steps of:
(a) Providing a Compound of Formula E: 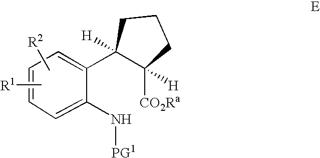
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group;

R^{a} is hydrogen or a suitable carboxyl protecting group, and

(b) Deprotecting and Cyclizing a Compound of Formula E to Form a Compound of Formula D.

As defined above, the PG¹ group of formula E is a suitable amino protecting group. Suitable amino protecting groups are well known in the art and include those described in detail in Greene (1999). Suitable amino protecting groups, taken with the —NH— moiety to which they are attached, include, but are not limited to, aralkylamines, carbamates, allyl amines, amides, and the like. In certain embodiments, the PG¹ group of formula E is selected from t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, or pivaloyl. In other embodiments, the PG¹ group of formula E is selected from t-butyloxycarbonyl, ethyloxycarbonyl, pivaloyl, or acetyl. In other embodiments, the PG¹ group of formula E is pivaloyl.

As defined above, the R^{a} group of formula E is either hydrogen or a suitable carboxyl protecting group. Suitable carboxyl protecting groups are well known in the art and include those described in detail in Greene (1999). Suitable carboxyl protecting groups, taken with the carboxylate moiety to which they are attached, include, but are not limited to, optionally substituted C₁₋₆ aliphatic esters, optionally substituted aryl esters, silyl esters, activated esters, amides, hydrazides, and the like. In certain embodiments, the R^{a} group of formula E is selected from methyl, ethyl, propyl, isopropyl, butyl, isobutyl, benzyl, and phenyl, wherein each group is optionally substituted. Additional suitable protected carboxylic acids include oxazolines and ortho esters. In certain embodiments, the R^{a} group of formula E is methyl, ethyl, or benzyl. In other embodiments, the R^{a} group of formula E is ethyl. In yet other embodiments, the R^{a} group of formula E is hydrogen.

Where the R^{a} group of formula E is a suitable carboxyl protecting group, one of ordinary skill in the art would recognize that, depending on the choice of PG¹ and R^{a}, cyclization may be performed prior to cleavage of one or both of these protecting groups. Similarly, cleavage of one or both of these protecting groups may occur concomitant with cyclization. Alternatively, cleavage of PG¹ may be performed following cyclization. One of ordinary skill in the art would also recognize that, depending on the presence or absence of, and the choice of, PG¹ and R^{a}, lactam formation may occur spontaneously. One of ordinary skill in the art would also appreciate that lactam formation may be induced thermally, with base catalysis, or with acid (Lewis or Bronsted) catalysis.

In certain embodiments, cyclization is performed with base catalysis. In other embodiments, cyclization is performed with acid catalysis. In certain embodiments, cyclization is performed in the presence of aqueous H₂SO₄. In other embodiments, the cyclization is performed with heating. In certain embodiments, the cyclization is performed with heating to about 85° C. In still other embodiments, the cyclization is performed with heating in the presence of an acid. In certain embodiments the cyclization is conducted at about 85° C. in the presence of aqueous H₂SO₄.

In certain embodiments, the present invention provides a method for preparing a compound of formula E: 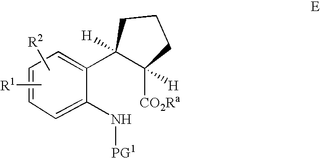
in enantiomerically enriched form, wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group, comprising the steps of:
(a) Providing a Compound of Formula F: 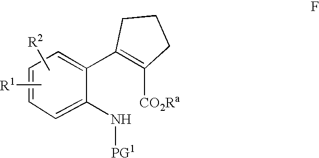
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group, and

(b) Hydrogenating Said Compound of Formula F in an Asymmetric Fashion to Provide Said Compound of Formula E in Enantiomerically Enriched Form.

As defined above, the PG¹ group of formula F is a suitable amino protecting group. Suitable amino protecting groups are well known in the art and include those described in detail in Greene (1999). Suitable amino protecting groups, taken with the —NH— moiety to which they are attached, include, but are not limited to, aralkylamines, carbamates, allyl amines, amides, and the like. In certain embodiments, the PG¹ group of formula F is selected from t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, or pivaloyl. In other embodiments, the PG¹ group of formula F is selected from t-butyloxycarbonyl, ethyloxycarbonyl, pivaloyl, or acetyl. In other embodiments, the PG¹ group of formula F is pivaloyl.

In certain embodiments, the asymmetric hydrogenation is catalyzed by a suitable chiral catalyst. In certain embodiments, the chiral catalyst is a complex comprising a transition metal species and a suitable chiral ligand. In certain embodiments, the transition metal species is a late transition metal species selected from a Ru, Rh, Pd, Ir, or Pt species. In other embodiments the transition metal species is a rhodium or ruthenium species.

In certain embodiments, the chiral catalyst is a pre-formed complex between a chiral ligand and a suitable transition metal species. In certain embodiments, where the chiral catalyst is a pre-formed complex between a chiral ligand and a suitable transition metal species, the chiral catalyst is selected from [((R,R)-Me-Butiphane)-Rh(COD)]BF₄ or [((S,S)-Me-MalPhos)-Rh(COD)]BF₄ (see Table I).

In certain embodiments, the chiral catalyst is formed by combining a chiral ligand with a suitable transition metal species. In embodiments wherein the chiral catalyst is formed by combining a chiral ligand with a suitable transition metal species, the transition metal species is selected from Rh(COD)₂SO₃CF₃ (where “COD” represents 1,5-cyclooctadiene), Rh(NOR)₂BF₄ (where “NOR” represents bicyclo[2.2.1]-hepta-2,5-diene, i.e., norbomadiene), Ru(COD)(Me-allyl)₂ (where “Me-allyl” represents 2-methylallyl), [RuC₂(p-cymene)]₂, or [RuCl₂(C₆H₆)]₂. In other embodiments wherein the chiral catalyst is formed by combining a chiral ligand with a suitable transition metal species, the transition metal species is selected from Rh(COD)₂SO₃CF₃ or Rh(NOR)₂BF₄. In certain embodiments wherein the chiral catalyst is formed by combining a chiral ligand with a suitable transition metal species, the chiral ligand contains a phosphorus moiety (e.g., a phosphine or phosphite moiety). In other embodiments the chiral ligand contains an olefinic moiety that is capable of binding a transition metal species. In yet other embodiments, the chiral ligand contains a carbene moiety that is capable of binding to a transition metal species. Suitable chiral ligands for asymmetric hydrogenation are well known in the art; see, e.g., Stereochemistry of Organic Compounds, E. L. Eliel and S. H. Silen, 1994, John Wiley and Sons; Asymmetric Catalysis in Organic Synthesis, R. Noyori, 1994, John Wiley and Sons; X. Cui and K. Burgess, Chem. Rev. 105:3272-3296 (2005); and W. Tang and X. Zhang, Chem. Rev. 103:3029-3069 (2003). Such chiral ligands are commercially available from, for example, Solvias or Sigma-Aldrich, or are prepared by methods known to one of ordinary skill in the art. Additional exemplary chiral ligands include, but are not limited to, JosiPhos-type, MandyPhos™-type, WalPhos-type, TaniaPhos™-type, RoPhos-type, DIPAMP-type, Butiphane-type, BPE-type, QUINAP-type, BINAP-type, NorPhos-type, MonoPhos™-type, TunePhos-type, MalPhos-type, DuPhos-type, PHOX-type, KetalPhos-type, f-KetalPhos-type, TangPhos-type, BIPHEP-type, ferrotane-type, Binaphane-type, f-Binaphane-type, Binapine-type, FAP-type, MOP-type, DIOP-type, ChiraPhos-type, BPPM-type, and BICP-type. In certain embodiments, the chiral ligand is a JosiPhos-type or a TaniaPhos™-type.

In certain embodiments, the chiral ligand is selected from those depicted in Table I. In other embodiments, the chiral ligand is selected from JosiPhos J002-1, J002-2, J216-1, J216-2, J202-2, or TaniaPhos T001-1 (see Table I). In yet other embodiments, the chiral ligand is JosiPhos J216-1 or J216-2 (see Table I).

TABLE I
Representative catalysts:

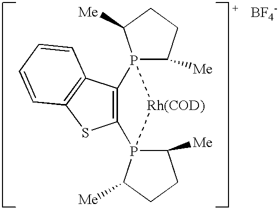

[((R,R)-Me-Butiphane)-Rh(COD)]BF₄

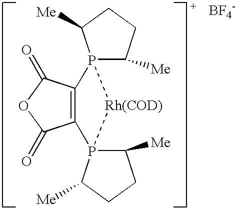

[((S,S)-Me-MalPhos)-Rh(COD)]BF₄
Representative ligands:

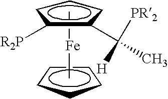

JosiPhos-type

| | |
|---|---|
| J001-1: | R = Ph, R′ = cyclohexyl |
| J002-1: | R = Ph, R′ = t-Bu |
| J002-2: | R = Ph, R′ = t-Bu [stereochemistry opposite to that depicted] |
| J003-1: | R = cyclohexyl, R′ = cyclohexyl |
| J005-1: | R = Ph, R′ = 3,5-dimethyiphenyl |
| J006-1: | R = 3,5-di(trifluoromethyl)phenyl, R′ = cyclohexyl |
| J007-1: | R = 3,5-dimethyl-4-methoxyphenyl, R′ = cyclohexyl |
| J008-1: | R = 3,5-di(trifluoromethyl)phenyl, R′ = 3,5-dimethyiphenyl |
| J009-1: | R = cyclohexyl, R′ = t-Bu |
| J011-1: | R = 4-trifluoromethylphenyl, R′ = t-Bu |
| J012-1: | R = para-tolyl, R′ = t-Bu |
| J013-1: | R = 3,5-dimethyl-4-methoxyphenyl, R′ = t-Bu |
| J015-2: | R = 2-furyl, R′ = cyclohexyl [stereochemistry opposite to that |
| | depicted] |
| J031-1: | R = phenyl, R′ = cyclopentyl |
| J202-2: | R = 4-methoxyphenyl, R′ = t-Bu [stereochemistry opposite to |
| | that depicted] |
| J211-1: | R = 2-methylphenyl, R′ t-Bu |
| J212-2: | R = 2-furyl, R′ = t-Bu [stereochemistry opposite to that |
| | depicted] |
| J216-1: | R = 1-naphthyl, R′ = t-Bu |
| J216-2: | R = 1-naphthyl, R′ = t-Bu [stereochemistry opposite to that |
| | depicted] |

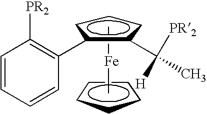

WallPhos-type
W003-1: R = Ph, R′ = cyclohexyl
W006-1: R = Ph, R′ = 3,5-dimethyiphenyl
W008-1: R = cyclohexyl, R′ = 3,5-di(trifluoromethyl)phenyl

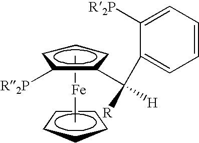

TaniaPhosTM-type
T001-1: R = dimethyamino, R′ = Ph, R″ = Ph
T002-1: R = dimethyamino, R′ = cyclohexyl, R″ = cyclohexyl

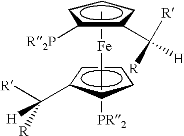

MandyPhosTM-type

| | |
|---|---|
| M001-1: | R = dimethyamino, R′ = Ph, R″ = Ph |
| M002-1: | R = dimethyamino, R′ = Ph, R″ = cyclohexyl |
| M004-1: | R = dimethyamino, R′ = Ph, |
| | R″ = 3,5-dimethyl-4-methoxyphenyl |

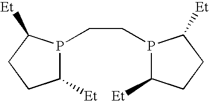

(R,R)-Et-BPE

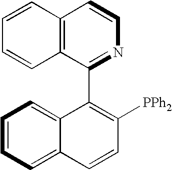

(R)-QUINAP

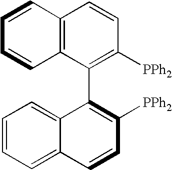

(S)-BINAP

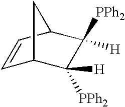

(+)-NorPhos

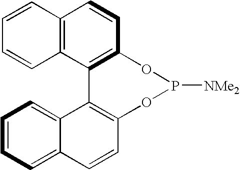

(R)-MonoPhos ™

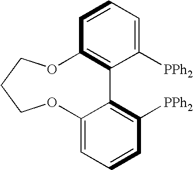

(R)-C3-TunePhos

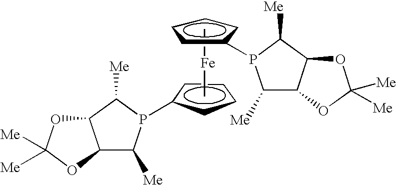

(S,S,S,S)-Me-f-KetalPhos

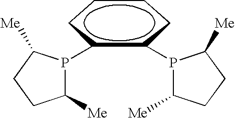

(S,S)-Me-DuPhos

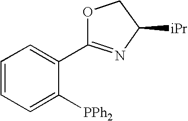

(R)-iPr-PHOX

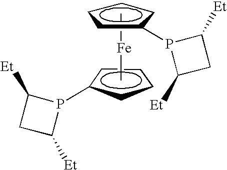

(R,R)-Et-FerroTane

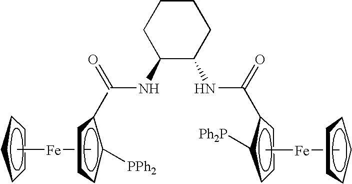

(SSS,S)-FAP

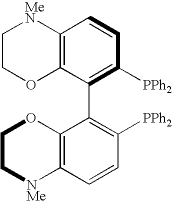

(R)-SolPhos

As defined above, the R^{a} group of formula F is either hydrogen or a suitable carboxyl protecting group. One of ordinary skill in the art would recognize that the CO₂R^{a} moiety of formula F may be in protected form (i.e., wherein R^{a} is a suitable carboxyl protecting group) during hydrogenation, or may be the free carboxylic acid (i.e., wherein R^{a} is hydrogen). In certain embodiments, R^{a} is hydrogen during hydrogenation. In other embodiments, R^{a} is a suitable carboxyl protecting group during hydrogenation. Suitable carboxyl protecting groups are well known in the art and include those described in detail in Greene (1999). Suitable carboxyl protecting groups, taken with the carboxylate moiety to which they are attached, include, but are not limited to, optionally substituted C₁₋₆ aliphatic esters, optionally substituted aryl esters, silyl esters, activated esters, amides, hydrazides, and the like. In certain embodiments, the PG² group in compounds of formula F is selected from methyl, ethyl, propyl, isopropyl, butyl, isobutyl, benzyl, and phenyl ester, wherein each group is optionally substituted. Additional suitable protected carboxylic acids include oxazolines and ortho esters. In certain embodiments, the PG² group of formula F is methyl, ethyl, or benzyl. In other embodiments, the PG² group of formula F is ethyl.

One of ordinary skill in the art would also recognize that wherein R^{a} is a protecting group that may be cleaved by hydrogenation, that cleavage of R^{a} and asymmetric hydrogenation of the cyclopentenyl olefin may occur concomitantly.

In certain embodiments R^{a} is a suitable carboxyl protecting group which is removed prior to hydrogenation. In other embodiments R^{a} is a base-cleavable protecting group which is removed prior to hydrogenation. In still other embodiments R^{a} is an ethyl group which is removed prior to hydrogenation. In yet other embodiments R^{a} is an ethyl group which is cleaved by treatment with KOH prior to hydrogenation.

In certain embodiments, the asymmetric hydrogenation is conducted at elevated temperatures. In other embodiments, the reaction is conducted at a temperature between about 30° C. and about 80° C. In still other embodiments, the reaction is conducted at a temperature of about 30° C. In yet other embodiments, the reaction is conducted at a temperature of about 50° C. In still other embodiments, the reaction is conducted at a temperature of about 80° C.

In certain embodiments, the asymmetric hydrogenation may be conducted at elevated pressures. In certain embodiments, the reaction is conducted at a pressure between about 225 psi and 450 psi. In other embodiments, the reaction is conducted at a pressure of about 225 psi. In yet other embodiments, the reaction is conducted at a pressure of about 450 psi.

In certain embodiments, the asymmetric hydrogenation provides a compound of formula E as a mixture of enantiomers in which one of the enantiomers comprises at least 25% of the mixture. In other embodiments, the asymmetric hydrogenation provides a compound of formula E as a mixture of enantiomers in which one of the enantiomers comprises at least 50% of the mixture. In still other embodiments, the asymmetric hydrogenation provides a compound of formula E as a mixture of enantiomers in which one of the enantiomers comprises at least 75% of the mixture. In yet other embodiments, the asymmetric hydrogenation provides a compound of formula E as a mixture of enantiomers in which one of the enantiomers comprises at least 90% of the mixture. In still other embodiments, the asymmetric hydrogenation provides a compound of formula E as a mixture of enantiomers in which one of the enantiomers comprises at least 95% of the mixture. In other embodiments, the asymmetric hydrogenation provides a compound of formula E as a mixture of enantiomers in which one of the enantiomers comprises at least 98% of the mixture. In certain embodiments, one enantiomer of a compound of formula E is provided substantially free from other stereoisomers.

According to another embodiment, the present invention provides a method of obtaining a compound of formula F: 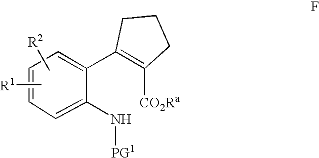
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group, comprising the steps of:
(a) Providing a Compound of Formula H: 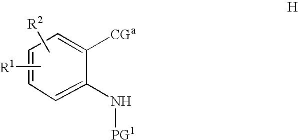
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group;

CG^{a} is a coupling group that facilitates transition metal-mediated Cₛₚ₂-Cₛₚ₂ coupling between the attached Cₛₚ₂ carbon and a Cₛₚ₂ carbon bearing a CG^{b} coupling group, and
(b) Coupling Said Compound of Formula H with a Compound of Formula G: 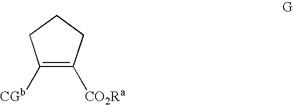
wherein:

CG^{b} is a coupling group that facilitates transition metal-mediated Cₛₚ₂-Cₛₚ₂ coupling between the attached Cₛₚ₂ carbon and a Cₛₚ₂ carbon bearing a CG^{a} coupling group; and

R^{a} is hydrogen or a suitable carboxyl protecting group, in the presence of a suitable transition metal.

In this portion of the invention, a compound of formula H is coupled to a compound of formula G, via a Cₛₚ₂-Cₛₚ₂ coupling reaction between the carbon centers bearing complementary coupling groups CG^{a} and CG^{b}. Suitable coupling reactions are well known to one of ordinary skill in the art and typically involve one of the coupling groups being an electron-withdrawing group (e.g., Cl, Br, I, OTf, etc.), such that the resulting polar carbon-CG bond is susceptible to oxidative addition by an electron-rich metal (e.g., a low-valent palladium species), and the complementary coupling group being an electropositive group (e.g., boronic acids, boronic esters, boranes, stannanes, silyl species, zinc species, aluminum species, magnesium species, zirconium species, etc.), such that the carbon which bears the electropositive coupling group is susceptible to transfer to other electropositive species (e.g., a Pd^{II-IV} species or a Ni^{II-IV} species). Exemplary reactions include those described in de Meijere (2004). In certain embodiments, CG^{a} in compounds of formula H is a boronic acid, a boronic ester, or a borane. In other embodiments, CG^{a} in compounds of formula H is a boronic ester. In still other embodiments, CG^{a} in compounds of formula H is a boronic acid. In certain embodiments, CG^{b} in compounds of formula G is Br, I, or OTf. In other embodiments, CG^{b} in compounds of formula G is OTf. In certain embodiments, the transformation is catalyzed by a palladium species. In other embodiments, the transformation is catalyzed by Pd(PPh₃)₄. In other embodiments, the reaction is performed as described in Jaroch et al., U.S. Pat. No. 6,391,887, Example 15.

The PG¹ group of formula H is a suitable amino protecting group. Suitable amino protecting groups are well known in the art and include those described in detail in Greene (1999). Suitable amino protecting groups, taken with the —NH— moiety to which they are attached, include, but are not limited to, aralkylamines, carbamates, allyl amines, amides, and the like. In certain embodiments, the PG¹ group of formula H, is selected from t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, or pivaloyl. In other embodiments, the PG¹ group of formula H, is selected from t-butyloxycarbonyl, ethyloxycarbonyl, pivaloyl, or acetyl. In still other embodiments, the PG¹ group of formula H is pivaloyl.

The R^{a} group in compounds of formula G is either hydrogen or a suitable carboxyl protecting group. In certain embodiments, the R^{a} group in compounds of formula G is hydrogen. In other embodiments, the R^{a} group in compounds of formula G is a suitable carboxyl protecting group. Suitable carboxyl protecting groups are well known in the art and include those described in detail in Greene (1999). Suitable carboxyl protecting groups, taken with the carboxylate moiety to which they are attached, include, but are not limited to, optionally substituted C₁₋₆ aliphatic esters, optionally substituted aryl esters, silyl esters, activated esters, amides, hydrazides, and the like. In certain embodiments, the R^{a} group of formula G is selected from methyl, ethyl, propyl, isopropyl, butyl, isobutyl, benzyl, and phenyl, wherein each group is optionally substituted. Additional suitable protected carboxylic acids include oxazolines and ortho esters. In other embodiments, the R^{a} group of formula G is methyl, ethyl, or benzyl. In other embodiments, the R^{a} group of formula G is ethyl.

In certain embodiments, the present invention provides a method for preparing a compound of formula E: 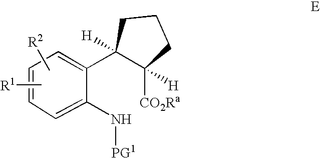
in enantiomerically enriched form, wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group, comprising the steps of:
(a) Providing a Compound of Formula H: 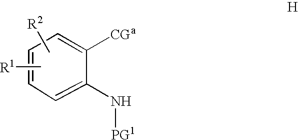
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group;

CG^{a} is a coupling group that facilitates transition metal-mediated Cₛₚ₂-Cₛₚ₂ coupling between the attached Cₛₚ₂ carbon and a Cₛₚ₂ carbon bearing a CG^{b} coupling group,
(b) Coupling Said Compound of Formula H with a Compound of Formula G: 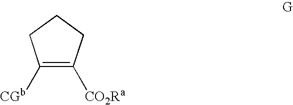
wherein:

CG^{b} is a coupling group that facilitates transition metal-mediated Cₛₚ₂-Cₛₚ₂ coupling between the attached Cₛₚ₂ carbon and a Cₛₚ₂ carbon bearing a CG^{a} coupling group; and

R^{a} is hydrogen or a suitable carboxyl protecting group, by the action of a suitable transition metal to provide a compound of formula F: 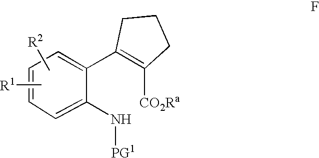
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG₁ is a suitable amino protecting group;

R^{a} is hydrogen or PG²; and

PG² is a suitable carboxyl protecting group, and

(c) Hydrogenating Said Compound of Formula F in an Asymmetric Fashion to Provide Said Compound of Formula E in Enantiomerically Enriched Form.

According to another embodiment, the present invention provides a method of obtaining a compound of formula D: 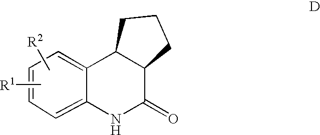
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, comprising the steps of:
(a) Providing a Compound of Formula F: 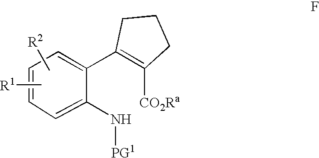
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group,
(b) Hydrogenating Said Compound of Formula F in an Asymmetric Fashion to Provide a Compound of Formula E: 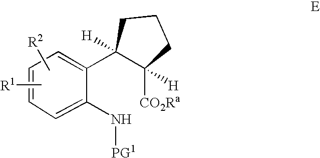
in enantiomerically enriched form, wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group, and

(c) Deprotecting and Cyclizing Said Compound of Formula E to Form Said Compound of Formula D.

Yet another aspect of the present invention provides a method for preparing a compound of formula C: 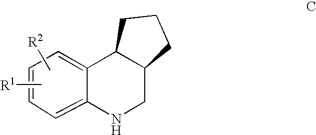
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, comprising the steps of:
(a) Providing a Compound of Formula F: 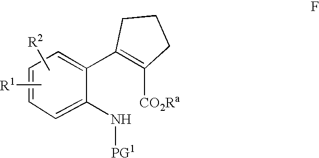
wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group,
(b) Hydrogenating Said Compound of Formula F in an Asymmetric Fashion to Provide a Compound of Formula E: 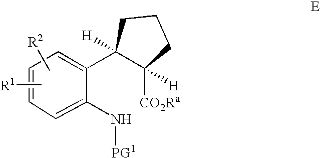
in enantiomerically enriched form, wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group,
(c) Deprotecting and Cyclizing a Compound of Formula E to Form a Compound of Formula D: 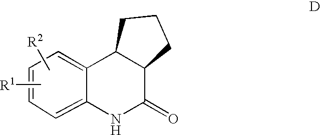
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, and

(d) Treating Said Compound of Formula D with a Suitable Reducing Agent to Form a Compound of Formula C.

Yet another aspect of the present invention provides a method for preparing a compound of formula C: 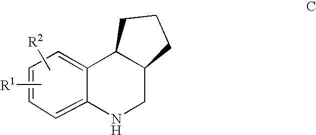
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, comprising the steps of:
(a) Providing a Compound of Formula E: 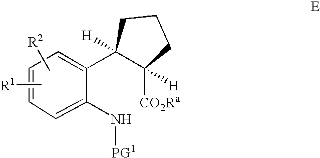
in enantiomerically enriched form, wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group,
(b) Deprotecting and Cyclizing a Compound of Formula E to Form a Compound of Formula D: 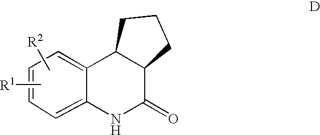
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group, and

(c) Treating Said Compound of Formula D with a Suitable Reducing Agent to Form a Compound of Formula C.

Another aspect of the present invention provides a compound of formula D: 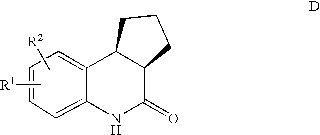
wherein:

R¹ and R² are each independently halogen, —CN, phenyl, —R, —OR, —C₁₋₆ perfluoroalkyl, or —OC₁₋₆ perfluoroalkyl; and

each R is independently hydrogen or a C₁₋₆ alkyl group.

In certain embodiments, one of the R¹ and R² groups of formula D is hydrogen. In another embodiment, both of the R¹ and R² groups of formula D are hydrogen. In yet another embodiment, one of the R¹ and R² groups of formula D is selected from halogen or —CN. In still another embodiment, one of the R¹ and R² groups of formula D is —OR, wherein R is hydrogen or a C₁₋₆ alkyl group. In another embodiment, one of the ¹ and R² groups of formula D is selected from —C₁₋₆ alkyl, —C₁₋₆ perfluoroalkyl, or —OC₁₋₆ perfluoroalkyl.

One of ordinary skill in the art will recognize that the asymmetric hydrogenation conditions providing compounds of formula E in enantiomerically enriched form can be adapted to provide compounds of formula E with stereochemistry opposite to that depicted in formula E. Accordingly, structures depicted herein are also meant to include compounds of formulae E, D, C, B, A, and II with stereochemistry opposite to that depicted. Additionally, one of ordinary skill in the art will recognize that the compounds of formulae D, C, B, A, and II which are formed from compounds of formula E are formed in enantiomerically enriched form as described in herein in embodiments describing compounds of formula E.

Yet another aspect of the present invention provides a compound of formula E: 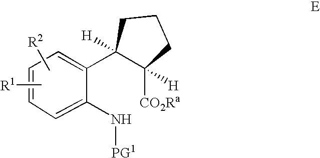
in enantiomerically enriched form, wherein:

R¹ and R² are each independently halogen, —CN, phenyl, —R, —OR, —C₁₋₆ perfluoroalkyl, or —OC₁₋₆ perfluoroalkyl;

each R is independently hydrogen or a C₁₋₆ alkyl group;

PG¹ is a suitable amino protecting group; and

R^{a} is hydrogen or a suitable carboxyl protecting group.

As defined above, the PG¹ group of formula E is a suitable amino protecting group. Suitable amino protecting groups are well known in the art and include those described in detail in Greene (1999). Suitable amino protecting groups, taken with the —NH— moiety to which they are attached, include, but are not limited to, aralkylamines, carbamates, allyl amines, amides, and the like. In certain embodiments, the PG¹ group of formula E is selected from t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, or pivaloyl. In other embodiments, the PG¹ group of formula E is selected from t-butyloxycarbonyl, ethyloxycarbonyl, pivaloyl, or acetyl. In other embodiments, the PG¹ group of formula E is pivaloyl.

As defined above, the R^{a} group of formula E is either hydrogen or a suitable carboxyl protecting group. Suitable carboxyl protecting groups are well known in the art and include those described in detail in Greene (1999). Suitable carboxyl protecting groups, taken with the carboxylate moiety to which they are attached, include, but are not limited to, optionally substituted C, aliphatic esters, optionally substituted aryl esters, silyl esters, activated esters, amides, hydrazides, and the like. In certain embodiments, the R^{a} group of formula E is selected from methyl, ethyl, propyl, isopropyl, butyl, isobutyl, benzyl, and phenyl, wherein each group is optionally substituted. Additional suitable protected carboxylic acids include oxazolines and ortho esters. In certain embodiments, the R^{a} group of formula E is methyl, ethyl, or benzyl. In other embodiments, the R^{a} group of formula E is ethyl. In yet other embodiments, the R^{a} group of formula E is hydrogen.

In certain embodiments, one of the R¹ and R² groups of formula E is hydrogen. In another embodiment, both of the R¹ and R² groups of formula E are hydrogen. In yet another embodiment, one of the R¹ and R² groups of formula E is selected from halogen or —CN. In still another embodiment, one of the R¹ and R² groups of formula E is —OR, wherein R is hydrogen or a C₁₋₆ alkyl group. In another embodiment, one of the R¹ and R² groups of formula E is selected from —C₁₋₆ alkyl, —C₁₋₆ perfluoroalkyl, or —OC₁₋₆ perfluoroalkyl.

In certain embodiments, the asymmetric hydrogenation provides a compound of formula E as a mixture of enantiomers in which one of the enantiomers comprises at least 25% of the mixture. In other embodiments, the asymmetric hydrogenation provides a compound of formula E as a mixture of enantiomers in which one of the enantiomers comprises at least 50% of the mixture. In still other embodiments, the asymmetric hydrogenation provides a compound of formula E as a mixture of enantiomers in which one of the enantiomers comprises at least 75% of the mixture. In yet other embodiments, the asymmetric hydrogenation provides a compound of formula E as a mixture of enantiomers in which one of the enantiomers comprises at least 90% of the mixture. In still other embodiments, the asymmetric hydrogenation provides a compound of formula E as a mixture of enantiomers in which one of the enantiomers comprises at least 95% of the mixture. In other embodiments, the asymmetric hydrogenation provides a compound of formula E as a mixture of enantiomers in which one of the enantiomers comprises at least 98% of the mixture. In certain embodiments, one enantiomer of a compound of formula E is provided substantially free from other stereoisomers.

### EXAMPLES

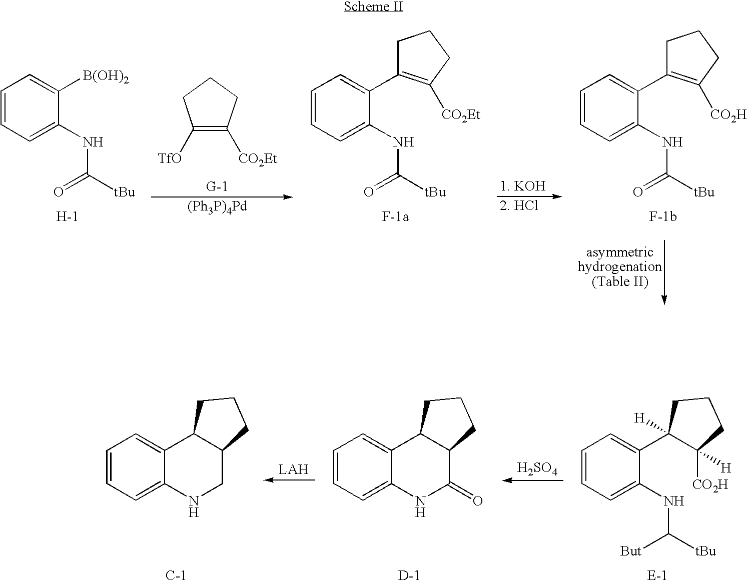

As indicated herein, the % ee data and the % conversion data for E-1 and ent-E-1 was obtained via the following chiral HPLC method:
Column: Chiracell OJ-RH
Solvent system: 75:25
95% water, 5% acetonitrile, 0.01% H₃PO₄: 95% acetonitrile, 5% water, 0.01%
H₃PO₄
Rate: 1 ml/min
Run time: 10 mins.
Wavelength: 210 nm

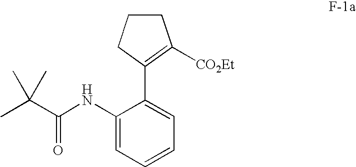

### 2-[2-(2,2-Dimethyl-propionylamino)-phenyl]-cyclopent-l-enecarboxylic acid ethyl ester (F-1a)

To a solution of 2-(N-pivaloylamino)phenylboronic acid (30.4 g, 0.14 mol, H-1) and 2-trifluoromethanesulfonyloxy-cyclopent-1-enecarboxylic acid ethyl ester (43.8 g, 0.15 mol, G-1) in 430 mL of dimethoxyethane (DME) was added 152 mL of water followed by potassium carbonate (30.4 g, 0.23 mol). The solution was thoroughly purged with nitrogen and then warmed to 60° C. A solution of tetrakis(triphenylphosphine)palladium (0.91 g, 0.79 mmol) in 60 mL DME was then added in a single portion. The mixture was then heated to 80° C. After 1 hr, the mixture was cooled to room temperature, treated with charcoal (Darco, G60, 100 mesh), and stirred for 20 minutes. The mixture was then filtered through a 1-2 cm pad of Celite® and rinsed with additional DME. The filtrate was concentrated to about ⅛ volume at which time crystals formed. Filtration and in vacuo drying afforded the title compound (F-1a) as an off-white solid (42.6 g, 98%). H¹ NMR (CDCl₃): 8.01 (d, 1H), 7.89 (br s, 1H), 7.5-7.1 (m, 3H), 4.13 (q, 2H), 2.94-2.80 (m, 4H), 2.08 (p, 2H), 1.29 (S, 9H), 1.12 (t, 3H).

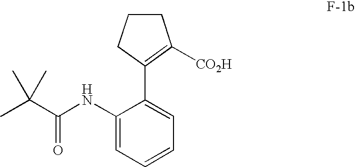

### 2-[2-(2,2-Dimethyl-propionylamino)-phenyl]-cyclopent-1-enecarboxylic acid (F-1b)

The ethyl ester from above (42.6 g, 0.14 mol, F-1a) was added to a mixture of 185 mL 1N KOH (aqueous) and 21 mL of tetrahydrofuran (THF). The mixture was heated to 70° C. for 5 hours at which time the mixture was cooled and then washed twice with ethyl acetate. The aqueous layer was treated with charcoal (Darco, G60, 100 mesh) and stirred for 30 minutes. The mixture was filtered through 1 cm of Celite® and rinsed with water. The filtrate was acidified with 1N HCl (aqueous) to a pH of 4-5. The solids that formed were filtered and rinsed thoroughly with water. The product (F-1b) was obtained as white granular crystals (32.4 g, 83%). H¹ NMR (CDCl₃): 7.73 (d/br s 2H overlap), 7.30 (t, 1H), 7.20 (t, 1H), 7.10 (d, 1H), 2.90-2.70 (m, 4H), 2.0 (p, 2H), 1.23 (s, 9H).

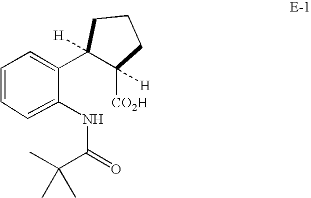

### 1(R),2(S)-[2-(2,2-Dimethyl-propionylamino)-phenyl]-cyclopentanecarboxylic acid (E-1)

A mixture of Rh(COD)₂SO₃CF₃ (0.7 mg, 1 mol %) and Josiphos SL-J202-2 (1.0 mg, 1 mol %) under inert atmosphere were combined with 1 mL of methanol and shaken for 60 minutes at ambient temperature. The mixture was added to 2-[2-(2,2-dimethyl-propionylamino)-phenyl]-cyclopent-1-enecarboxylic acid (50 mg, 0.174 mmol, F-1b) in 1 mL of methanol. The mixture was stirred under 450 psi H₂ at 50° C. for 36 hours. After cooling and purging with N₂, the mixture was concentrated in vacuo. HPLC analysis indicated complete conversion (100% yield of E-1) and chiral purity of 72% ee. H¹ NMR (CDCl₃): 7.48 (br s, 1H), 7.34 (d, 2H), 7.28 (m, 1H), 7.11 (d, 1H), 3.52-3.43 (m, 1H), 3.28-3.20 (m, 1H), 2.43-2.30 (m, 1H), 2.05-1.94 (m, 4H), 1.66-1.54 (m, 1H), 1.41 (s, 9H). Additional conditions for asymmetric hydrogenation are provided in Table II.

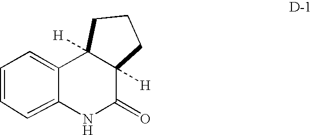

### 1,2,3,3a(R),5,9b(S)-Hexahydro-cyclopenta[c]quinolin-4-one (D-1)

1R,2S-[2-(2,2-Dimethyl-propionylamino)-phenyl]-cyclopentanecarboxylic acid (100 mg, 0.53 mmol, E-1) was dissolved in 0.25 mL of DME. 1.5 mL of 30% H₂SO₄ (aqueous) was then added and the mixture was heated to 85° C. After 10 hours, the reaction was cooled and then diluted with ethyl acetate. After washing with saturated aqueous NaHCO₃, the organics were dried over anhydrous MgSO₄. Filtration and concentration provided 51 mg of the title quinolinone compound (D-1). H¹ NMR (CDCl₃): 8.97 (br s, 1H), 7.23-7.15 (m, 2H), 7.01 (t, 1H), 6.83 (d, 1H), 3.27 (q, 1H), 3.01-2.94 (m, 1H), 2.36-2.30 (m, 1H), 2.18-2.07 (m, 2H), 1.76-1.64 (m, 3H).

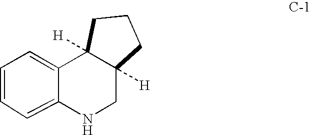

### 2,3,3a(S),4,5 9b(S)-Hexahydro-1H-cyclopentafclquinoline (C-1)

1,2,3,3a(R),5,9b(S)-Hexahydro-cyclopenta[c]quinolin-4-one (30 mg, 0.16 mmol) is combined with 0.6 mL anhydrous THF. Lithium aluminum hydride solution (0.6 mL, 1N in THF) was added dropwise. The mixture was stirred under nitrogen overnight. After quenching with 1N HCl (aqueous), the product (C-1)was extracted with ethyl acetate and dried over anhydrous MgSO₄. H¹ NMR (CDCl₃): 7.12 (d, 1H), 7.00 (t, 1H), 6.71 (t, 1H), 6.60 (d, 1H), 3.18-3.12 (dd, 1H), 3.02 (q, 1H), 2.84 (t, 1H), 2.45-2.33 (m, 1H), 2.26-2.14 (m, 1H), 2.08-1.95 (m, 1H), 1.80-1.40 (m, 4H).

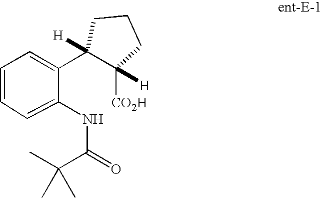

### 1S,2R-[2-(2,2-Dimethyl-propionylamino)-phenyl]-cyclopentanecarboxylic acid (ent-E-1)

A mixture of Rh(NOR)₂BF₄ (0.7 mg, 1 mol %) and Josiphos SL-J216-1 (1.1 mg, 1 mol %) under inert atmosphere were combined with 1 mL of methanol and shaken for 60 minutes at ambient temperature. The mixture was added to 2-[2-(2,2-Dimethyl-propionylamino)-phenyl]-cyclopent-1-enecarboxylic acid (50 mg, 0.174 mmol, F-1b) in 1 mL of methanol. The mixture was stirred under 450 psi H₂ at 80° C. for 24 hours. After cooling and purging with N₂, the mixture was concentrated in vacuo. HPLC analysis indicated complete conversion (100% yield) and chiral purity of 92% ee. H¹ NMR (CDCl₃): 7.48 (br s, 1H), 7.34 (d, 2H), 7.28 (m, 1H), 7.11 (d, 1H), 3.52-3.43 (m, 1H), 3.28-3.20 (m, 1H), 2.43-2.30 (m, 1H), 2.05-1.94 (m, 4H), 1.66-1.54 (m, 1H), 1.41 (s, 9H). Additional conditions for asymmetric hydrogenation are provided in Table II.

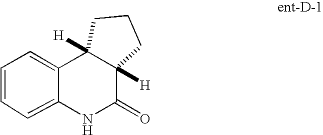

### 1,2,33a(S),5,9b(R)-Hexahydro-cyclopenta[c]quinolin-4-one (ent-D-1)

1S,2R-[2-(2,2-Dimethyl-propionylamino)-phenyl]-cyclopentanecarboxylic acid (100 mg, 0.53 mmol, ent-E-1) was dissolved in 0.25 mL of DME. 1.5 mL of 30% H₂SO₄ (aqueous) was then added and the mixture was heated to 85° C. After 10 hours, the reaction was cooled and then diluted with ethyl acetate. After washing with saturated aqueous NaHCO₃, the organics were dried over anhydrous MgSO₄. Filtration and concentration provided 51 mg of the title quinolinone (ent-D-1). H¹ NMR (CDCl₃): 8.97 (br s, 1H), 7.23-7.15 (m, 2H), 7.01 (t, 1H), 6.83 (d, 1H), 3.27 (q, 1H), 3.01-2.94 (m, 1H), 2.36-2.30 (m, 1H), 2.18-2.07 (m, 2H), 1.76-1.64 (m, 3H).

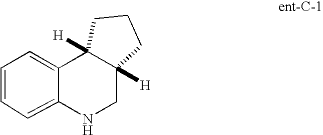

### 2,3,3a(R),4,5,9b(R)-Hexahydro-1H-cyclopenta[c]quinoline (ent-C-1)

1,2,3,3a(S),5,9b(R)-Hexahydro-cyclopenta[c]quinolin-4-one (30 mg, 0.16 mmol, ent-D-1) was combined with 0.6 mL of anhydrous THF. Lithium aluminum hydride solution (0.6 mL, 1N in THF) was added dropwise. The mixture was stirred under nitrogen overnight. After quenching with 1N HCl (aqueous), the product (ent-C-1) was extracted with ethyl acetate and dried over anhydrous MgSO₄. H¹ NMR (CDCl₃): 7.12 (d, 1H), 7.00 (t, 1H), 6.71 (t, 1H), 6.60 (d, 1H), 3.18-3.12 (dd, 1H), 3.02 (q, 1H), 2.84 (t, 1H), 2.45-2.33 (m, 1H), 2.26-2.14 (m, 1H), 2.08-1.95 (m, 1H), 1.80-1.40 (m, 4H).

In addition to the exemplary preparations of E-1 and ent-E-1 provided above, Table II lists combinations of chiral ligands and transition metals (entries 1-51, 53-57, and 59-68), as well as pre-formed chiral complexes (entries, 52 and 58), that were also found to be useful as catalysts in the asymmetic hydrogenation of cyclopentene F-1b to form E-1 as shown. Typically, the transition metal species and the chiral ligand were employed in a 1:1 ratio, except where the transition metal species was bimetallic, in which case the ratio was 1:2 (i.e., 1 ligand per metal canter). The chiral catalysts, including pre-formed chiral complexes, were typically emploted in a sucstrate (F-1b) to catalyst ratio of 100:1. Reactions were run as described above, with MeOH or THF as solvent, at temperatures ranging from about 30° C. to about 80° C., at pressures ranging from about 225 psi to about 450 psi, and optionally in the presence of triethylamine as an additive.

TABLE II

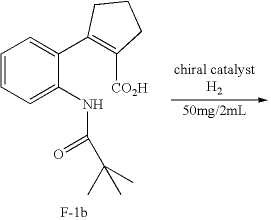

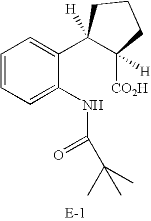

| entry | transition metal species | chiral ligand |
|---|---|---|
| 1 | Rh(COD)₂SO₃CF₃ | JosiPhos J0001-1 |
| 2 | Rh(COD)₂SO₃CF₃ | JosiPhos J002-1 |
| 3 | Rh(NOR)₂BF₄ | JosiPhos J002-1 |
| 4 | Ru(COD)(Me-allyl)₂ | JosiPhos J002-1 |
| 5 | Rh(COD)₂SO₃CF₃ | JosiPhos J002-2 |
| 6 | Rh(NOR)₂BF₄ | JosiPhos J002-2 |
| 7 | [RuCl₂(p-cymene)]₂ | JosiPhos J002-2 |
| 8 | Rh(COD)₂SO₃CF₃ | JosiPhos J003-1 |
| 9 | Rh(COD)₂SO₃CF₃ | JosiPhos J005-1 |
| 10 | Rh(COD)₂SO₃CF₃ | JosiPhos J006-1 |
| 11 | Rh(COD)₂SO₃CF₃ | JosiPhos J007-1 |
| 12 | Rh(COD)₂SO₃CF₃ | JosiPhos J008-1 |
| 13 | Rh(COD)₂SO₃CF₃ | JosiPhos J009-1 |
| 14 | Rh(NOR)₂BF₄ | JosiPhos J009-1 |
| 15 | Rh(COD)₂SO₃CF₃ | JosiPhos J011-1 |
| 16 | Rh(COD)₂SO₃CF₃ | JosiPhos J012-1 |
| 17 | Rh(COD)₂SO₃CF₃ | JosiPhos J013-1 |
| 18 | Rh(COD)₂SO₃CF₃ | JosiPhos J015-2 |
| 19 | Rh(COD)₂SO₃CF₃ | JosiPhos J031-1 |
| 20 | Rh(COD)₂SO₃CF₃ | JosiPhos J202-2 |
| 21 | Rh(NOR)₂BF₄ | JosiPhos J202-2 |
| 22 | Rh(COD)₂SO₃CF₃ | JosiPhos J211-1 |
| 23 | Rh(COD)₂SO₃CF₃ | JosiPhos J212-2 |
| 24 | Rh(COD)₂SO₃CF₃ | JosiPhos J216-1 |
| 25 | Rh(NOR)₂BF₄ | JosiPhos J216-1 |
| 26 | Ru(COD)(Me-allyl)₂ | JosiPhos J216-1 |
| 27 | Rh(NOR)₂BF₄ | JosiPhos J216-1 |
| 28 | [RuCl₂(C₆H₆)]2 | JosiPhos J216-1 |
| 29 | Rh(NOR)₂BF₄ | JosiPhos J216-1 |
| 30 | Rh(COD)₂SO₃CF₃ | WalPhos W003-1 |
| 31 | Rh(COD)₂SO₃CF₃ | WalPhos W006-1 |
| 32 | Rh(NOR)₂BF₄ | WalPhos W008-1 |
| 33 | Rh(COD)₂SO₃CF₃ | TaniaPhos T001-1 |
| 34 | Rh(NOR)₂BF₄ | TaniaPhos T001-1 |
| 35 | [RuCl₂(C₆H₆)]2 | TaniaPhos T001-1 |
| 36 | Rh(COD)₂SO₃CF₃ | TaniaPhos T002-1 |
| 37 | Rh(COD)₂SO₃CF₃ | MandyPhos M001-1 |
| 38 | Rh(COD)₂SO₃CF₃ | MandyPhos M002-1 |
| 39 | Rh(COD)₂SO₃CF₃ | MandyPhos M004-1 |
| 40 | Rh(COD)₂SO₃CF₃ | (R,R)-Et-BPE |
| 41 | Ru(COD)(Me-allyl)₂ | (R,R)-Et-BPE |
| 42 | [((RR)-Me-Butiphane)-Rh(COD)]BF₄ | |
| 43 | Rh(COD)₂SO₃CF₃ | (R)-QUINAP |
| 44 | Rh(COD)₂SO₃CF₃ | (S)-BINAP |
| 45 | Rh(COD)₂SO₃CF₃ | (+)-NorPhos |
| 46 | Rh(COD)₂SO₃CF₃ | (R)-MonoPhos |
| 47 | Rh(COD)₂SO₃CF₃ | (R)-C3-TunePhos |
| 48 | [((S,S)-Me-MalPhos)-Rh(COD)]BF₄ | |
| 49 | Rh(COD)₂SO₃CF₃ | (S,S)-Me-DuPhos |
| 50 | Ru(COD)(Me-allyl)₂ | (S,S)-Me-DuPhos |
| 51 | Rh(COD)₂SO₃CF₃ | (R)-iPr-PHOX |
| 52 | Rh(COD)₂SO₃CF₃ | (S,S,S,S)-Me-f- |
| | KetalPhos | |
| 53 | Rh(NOR)₂BF₄ | (S,S,S,S)-Me-f- |
| | KetalPhos | |
| 54 | Rh(COD)₂SO₃CF₃ | (R,R)-Et-FerroTane |
| 55 | Rh(COD)₂SO₃CF₃ | (S,S,S,S)-FAP |
| 56 | [RuCl₂(p-cymene)]₂ | (R)-SolPhos |
| 57 | Rh(NOR)₂BF₄ | JosiPhos J216-2 |

We claim:

1. A method for preparing a compound of formula II: 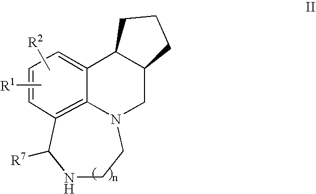
or a pharmaceutically acceptable salt thereof, wherein:
n is 0, 1, or 2;
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group; and
R⁷is —R, comprising the steps of:
(a) providing a compound of formula F: 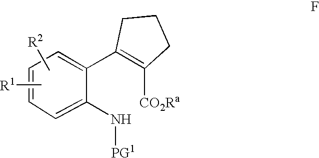
wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(b) hydrogenating said compound of formula F in an asymmetric fashion to provide a compound of formula E: 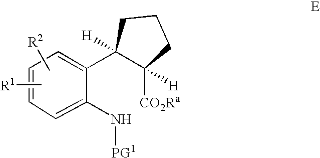
in enantiomerically enriched form, wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(c) deprotecting and cyclizing a compound of formula E to form a compound of formula D: 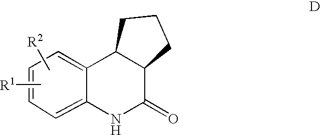
wherein:
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group,
(d) treating said compound of formula D with a suitable reducing agent to form a compound of formula C: 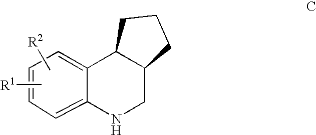
wherein:
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group,
(e) alkylating said compound of formula C to form a compound of formula B: 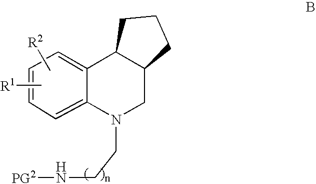
wherein:
n is 0, 1, or 2;
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group; and
PG² is a suitable amino protecting group,
(f) deprotecting said compound of formula B to form said compound of formula A: 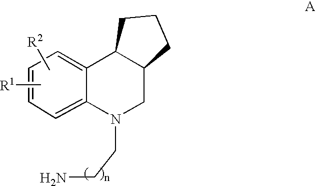
wherein:
n is 0, 1, or 2;
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group, and
(g) reacting said compound of formula A with an aldehyde of formula R⁷CHO, or a suitable formaldehyde equivalent, to form a compound of formula II.

2. The method according to claim 1, wherein each occurrence of n is 1 and each occurrence of R¹ and R² is hydrogen.

3. The method according to claim 1, wherein the PG¹ group of formulae F and E is pivaloyl, t-butyloxycarbonyl, ethyloxycarbonyl, or acetyl.

4. The method according to claim 1, wherein the R^{a} group of formulae F and E is hydrogen or ethyl.

5. The method according to claim 1, wherein the PG² group of formula B is acetyl.

6. The method according to claim 1, wherein the hydrogenation of said compound of formula F in an asymmetric fashion at step (b) is catalyzed by a chiral catalyst.

7. The method according to claim 6, wherein the chiral catalyst is a complex comprising a Ru, Rh, Pd, Ir, or Pt species and a suitable chiral ligand.

8. The method according to claim 1, wherein the deprotection and cyclization at step (c) is achieved by reacting said compound of formula E in the presence of aqueous H₂SO₄.

9. The method according to claim 1, wherein the suitable reducing agent at step (d) is selected from LiAlH₄, NaAlH₄, LiHAl(OMe)₃, BH₃, or NaBH₄.

10. The method according to claim 1, wherein the alkylation at step (e) is is achieved by reacting said compound of formula C with 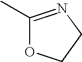
in the presence of a suitable acid to form a compound of formula B: 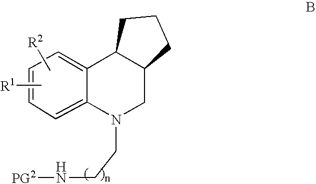
wherein n is 1 and PG² is acetyl.

11. The method according to claim 1, wherein the aldehyde of formula R⁷CHO at step (g) is formaldehyde or a suitable equivalent thereof.

12. A method for preparing a compound of formula C: 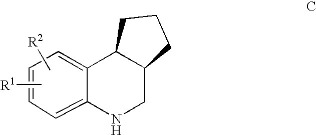
wherein:
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group, comprising the steps of:
(a) providing a compound of formula F: 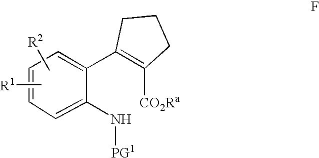
wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(b) hydrogenating said compound of formula F in an asymmetric fashion to provide a compound of formula E: 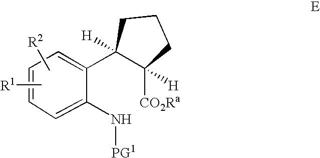
in enantiomerically enriched form, wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(c) deprotecting and cyclizing a compound of formula E to form a compound of formula D: 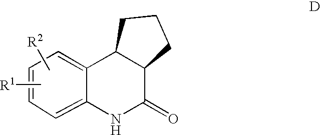
wherein:
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group,
(d) treating said compound of formula D with a suitable reducing agent to form a compound of formula C.

13. The method according to claim 12, wherein each occurrence of R¹ and R² is hydrogen.

14. The method according to claim 12, wherein the PG¹ group of formulae F and E is pivaloyl, t-butyloxycarbonyl, ethyloxycarbonyl, or acetyl.

15. The method according to claim 12, wherein the R^{a} group of formulae F and E is hydrogen or ethyl.

16. The method according to claim 12, wherein the PG² group of formula B is acetyl.

17. The method according to claim 12, wherein the hydrogenation of said compound of formula F in an asymmetric fashion at step (b) is catalyzed by a chiral catalyst.

18. The method according to claim 17, wherein the chiral catalyst is a complex comprising a Ru, Rh, Pd, Ir, or Pt species and a suitable chiral ligand.

19. The method according to claim 12, wherein the deprotection and cyclization at step (c) is achieved by reacting said compound of formula E in the presence of aqueous H₂SO₄.

20. The method according to claim 12, wherein the suitable reducing agent at step (d) is selected from LiAlH₄, NaAlH₄, LiHAl(OMe)₃, BH₃, or NaBH₄.

21. A method for preparing a compound of formula D: 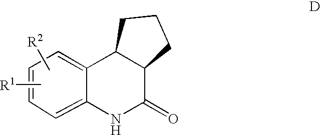
wherein:
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group, comprising the steps of:
(a) providing a compound of formula F: 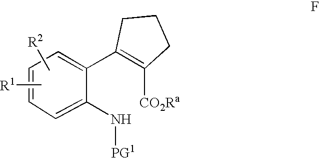
wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(b) hydrogenating said compound of formula F in an asymmetric fashion to provide a compound of formula E: 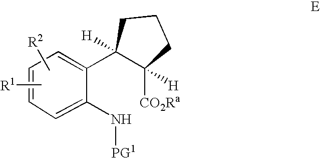
in enantiomerically enriched form, wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(c) deprotecting and cyclizing a compound of formula E to form a compound of formula D.

22. The method according to claim 21, wherein each occurrence of R¹ and R² is hydrogen.

23. The method according to claim 21, wherein the PG¹ group of formulae F and E is pivaloyl, t-butyloxycarbonyl, ethyloxycarbonyl, or acetyl.

24. The method according to claim 21, wherein the R^{a} group of formulae F and E is hydrogen or ethyl.

25. The method according to claim 21, wherein the hydrogenation of said compound of formula F in an asymmetric fashion at step (b) is catalyzed by a chiral catalyst.

26. The method according to claim 25, wherein the chiral catalyst is a complex comprising a Ru, Rh, Pd, Ir, or Pt species and a suitable chiral ligand.

27. The method according to claim 21, wherein the deprotection and cyclization at step (c) is achieved by reacting said compound of formula E in the presence of aqueous H₂SO₄.

28. A method for preparing a compound of formula E: 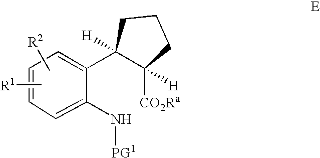
in enantiomerically enriched form, wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group, comprising the steps of:
(a) providing a compound of formula F: 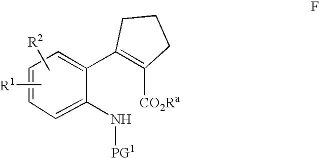
wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group,
(b) hydrogenating said compound of formula F in an asymmetric fashion to provide a compound of formula E.

29. The method according to claim 28, wherein each occurrence of R¹ and R² is hydrogen.

30. The method according to claim 28, wherein the PG¹ group of formulae F and E is pivaloyl, t-butyloxycarbonyl, ethyloxycarbonyl, or acetyl.

31. The method according to claim 28, wherein the R^{a} group of formulae F and E is hydrogen or ethyl.

32. The method according to claim 28, wherein the hydrogenation of said compound of formula F in an asymmetric fashion at step (b) is catalyzed by a chiral catalyst.

33. The method according to claim 28, wherein the chiral catalyst is a complex comprising a Ru, Rh, Pd, Ir, or Pt species and a suitable chiral ligand.

34. A compound of formula E: 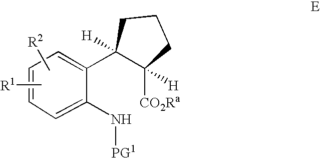
in enantiomerically enriched form, wherein:
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
PG¹ is a suitable amino protecting group; and
R^{a} is hydrogen or a suitable carboxyl protecting group.

35. The compound according to claim 34, wherein each of R¹ and R² is hydrogen, PG¹ is pivaloyl, t-butyloxycarbonyl, ethyloxycarbonyl, or acetyl, and R^{a} is hydrogen or ethyl.

36. The compound according to claim 35, wherein each of R¹ and R² is hydrogen, PG¹ is pivaloyl, and R^{a} is hydrogen.

37. A compound of formula D: 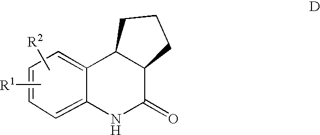
wherein:
R¹ and R² are each independently halogen, —CN, phenyl, —R, —OR, —C₁₋₆ perfluoroalkyl, or —OC₁₋₆ perfluoroalkyl; and
each R is independently hydrogen or a C₁₋₆ alkyl group.

38. The compound according to claim 37, wherein each of R¹ and R² is hydrogen.
